(19) United States
(12) Patent Application Publication
(10) Pub. No.: US 2017/0319706 A1
(43) Pub. Date: Nov. 9, 2017

(54) DELIVERING A DRUG TO A MUCOSAL SURFACE

(71) Applicant: Dow Global Technologies LLC, Midland, MI (US)

(72) Inventors: Jaime L. Curtis-Fisk, Midland, MI (US); Robert B. Appell, Midland, MI (US); Peter M. Margl, Midland, MI (US); Susan L. Jordan, Collegeville, PA (US); True L. Rogers, Midland, MI (US)

(21) Appl. No.: 15/526,027

(22) PCT Filed: Nov. 5, 2015
(86) PCT No.: PCT/US2015/059166

§ 371 Date: May 11, 2017

Related U.S. Application Data
(60) Provisional application No. 62/081,141, filed on Nov. 18, 2014.

(51) Int. Cl. A61K 47/61; A61K 31/14; A61K 9/08; C08B 15/06; A61K 9/00

(52) U.S. Cl. CPC A61K 47/61; C08B 15/06; A61K 9/0043; A61K 9/08; A61K 31/14

(57) ABSTRACT

Provided is a method of delivering a drug to a mucosal surface in a living body, said method comprising applying a solution to said mucosal surface, wherein said solution comprises a cationic polymer dissolved in water, wherein said cationic polymer comprises a cationic functional group covalently attached to a polysaccharide polymer backbone selected from the group consisting of amylodextrin polymers, methylcellulose polymers, and hydroxypropyl methylcellulose polymers.

Mucosal surfaces line various cavities in a living body, including those exposed to the external atmosphere. Mucosal surfaces are involved in absorption of compounds into the body; consequently a useful method of introducing a drug into the body is to apply a composition containing the drug to the mucosal surface. When applying such a composition to a mucosal surface, it is desirable that the composition reside on the mucosal surface for a relatively long time. To improve that residence time, the composition may include a compound (called an “excipient”) in addition to the drug. It is considered that residence time will be lengthened if the excipient has a strong interaction with mucin protein. An important mucosal surface in the human body for introduction of drugs is the mucosal surface inside the nasal cavity.

EP 0 590 655 describes using cationic polysaccharide polymers to treat infirmities of mucosal surfaces. Cationic-functional hydroxypropylmethyl cellulose is not disclosed among the cationic polysaccharide polymers described by EP 0 590 655. In the process of making the present invention, it was considered that hydroxypropylmethyl cellulose (HPMC) can be utilized as an additive to solutions that are applied to the mucosal surface of the nasal cavity, and that the HPMC provided one or more benefits to such solutions, for example increasing the residence time in the nasal cavity. It is desired to provide a cationic polymer that has both cationic functionality and one or more of the benefits of HPMC. It is also desired to provide cationic polymer that has cationic functionality and that has polymer backbone of either amylodextrin or methylcellulose.

The following is a statement of the invention.

An aspect of the present invention is a method of delivering a drug to a mucosal surface in a living body, said method comprising applying a solution to said mucosal surface, wherein said solution comprises a cationic polymer dissolved in water, wherein said cationic polymer comprises a cationic functional group covalently attached to a polysaccharide polymer backbone selected from the group consisting of amylodextrin polymers, methylcellulose polymers, and hydroxypropyl methylcellulose polymers.

The following is a detailed description of the invention.

As used herein, the following terms have the designated definitions, unless the context clearly indicates otherwise.

Mucosal surfaces are found in living bodies of animals and humans. Mucosal surfaces are covered in epithelium. Examples of mucosal surfaces are found in the nasal cavity, the mouth, the esophagus, the stomach, the intestines, and other parts of the body.

A “polymer,” as used herein is a relatively large molecule made up of the reaction products of smaller chemical repeat units. Polymers may have a single type of repeat unit (“homopolymers”) or they may have more than one type of repeat unit (“copolymers”). Copolymers may have the various types of repeat units arranged randomly, in sequence, in blocks, in other arrangements, or in any mixture or combination thereof. Polymers have weight-average molecular weight of 2,000 daltons or higher.

A compound is considered herein to be cationic if an atom or a chemical group that bears a positive charge is covalently bound to the compound. A cationic functional group is an atom or a chemical group that bears a positive charge.

An amount of polymer is considered herein to be dissolved in water if the mixture of that amount of the polymer and water forms a stable composition that is not hazy to the unaided eye and that does not show phase separation of the polymer from the water.

As used herein, a drug is a compound that can have a therapeutic effect in a living body.

The present invention involves a cationic polymer that contains a cationic functional group attached to a polysaccharide polymer backbone. That is, the cationic polymer has a structure that would result if a molecule of the polysaccharide polymer (the “backbone” polymer) were subjected to one or more chemical reactions to replace one of the hydrogen atoms on the polysaccharide polymer with a cationic functional group. Regardless of the method of making the cationic polymer, the cationic polymer can be characterized by the properties of the backbone polymer.

Suitable polysaccharide polymers are amylodextrin polymers, methylcellulose polymers, and hydroxypropylmethylcellulose polymers.

Methylcellulose (MC) polymer has the structure I:

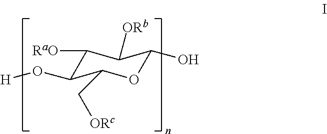

In structure I, the repeat unit is shown within the brackets. The index n is sufficiently large that structure I is a polymer. —R^{a}, —R^{b}, and —R^{c} is each independently chosen from —H and —CH₃. The choice of —R^{a}, —R^{b}, and —R^{c} may be the same in each repeat unit, or different repeat units may have different choices of —R^{a}, —R^{b}, and —R^{c}.

Methylcellulose polymer is characterized by the weight percent of methoxyl groups. The weight percentages are based on the total weight of the methylcellulose polymer. By convention, the weight percent is an average weight percentage based on the total weight of the cellulose repeat unit, including all substituents. The content of the methoxyl group is reported based on the mass of the methoxyl group (i.e., —OCH3). The determination of the % methoxyl in methylcellulose (MC) polymer is carried out according to the United States Pharmacopeia (USP 37, “Methylcellulose”, pages 3776-3778).

Methylcellulose polymer is also characterized by the viscosity of a 2 wt.-% solution in water at 20° C. The 2% by weight methylcellulose polymer solution in water is prepared and tested according to United States Pharmacopeia (USP 37, “Methylcellulose”, pages 3776-3778). As described in the United States Pharmacopeia, viscosities of less than 600 mPa·s are determined by Ubbelohde viscosity measurement and viscosities of 600 mPa·s or more are determined using a Brookfield viscometer. This viscosity is known herein as the “2% solution viscosity.”

Hydroxypropyl methylcellulose polymer has the structure I, where —R^{a}, —R^{b}, and —R^{c} is each independently chosen from —H, —CH₃, and structure II:

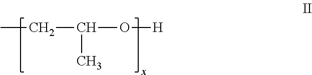

The choice of —R^{a}, —R^{b}, and —R^{c} may be the same in each repeat unit, or different repeat units may have different choices of —R^{a}, —R^{b}, and —R^{c}. The number x is an integer of value 1 or larger. One or more of —R^{a}, —R^{b}, and —R^{c} has structure II on one or more of the repeat units.

Hydroxypropyl methylcellulose polymer is characterized by the weight percent of methoxyl groups. The weight percentages are based on the total weight of the hydroxypropyl methylcellulose polymer. By convention, the weight percent is an average weight percentage based on the total weight of the cellulose repeat unit, including all substituents. The content of the methoxyl group is reported based on the mass of the methoxyl group (i.e., —OCH₃). The determination of the % methoxyl in hydroxypropyl methylcellulose polymer is carried out according to the United States Pharmacopeia (USP 37, “Hypromellose”, pages 3296-3298).

Hydroxypropyl methylcellulose polymer is characterized by the weight percent of oxyhydroxypropyl groups. The weight percentages are based on the total weight of the hydroxypropyl methylcellulose polymer. The content of the hydroxypropoxyl group is reported based on the mass of the hydroxypropoxyl group (i.e., —O—C₃H₆OH). The determination of the % hydroxypropoxyl in hydroxypropyl methylcellulose (HPMC) is carried out according to the United States Pharmacopeia (USP 37, “Hypromellose”, pages 3296-3298).

Hydroxypropylmethylcellulose polymer is also characterized by the viscosity of a 2 wt.-% solution in water at 20° C. The 2% by weight hydroxypropylmethylcellulose polymer solution in water is prepared and tested according to United States Pharmacopeia (USP 37, “Hypromellose”, pages 3296-3298). As described in the United States Pharmacopeia, viscosities of less than 600 mPa·s are determined by Ubbelohde viscosity measurement and viscosities of 600 mPa·s or more are determined using a Brookfield viscometer. This viscosity is known herein as the “2% solution viscosity.”

The category of polymers formed by combining methylcellulose polymers and hydroxypropyl methylcellulose polymers is known herein as “(HP)methylcellulose polymers.”

Preferred (HP)methylcellulose polymer backbone polymers are described as follows. Preferably, the weight percent of methoxyl groups is 15% or more; more preferably 20% or more; more preferably 25% or more. Preferably, the weight percent of methoxyl groups is 40% or less; more preferably 36% or less; more preferably 32% or less.

Preferably, (HP)methylcellulose polymer backbone polymers have 2% solution viscosity of 1.5 mPa-s or more; more preferably 2 mPa-s or more; more preferably 3 mPa-s or more; more preferably 4 mPa-s or more; more preferably 10 mPa-s or more; more preferably 30 mPa-s or more; more preferably 100 mPa-s or more; more preferably 300 mPa-s or more; more preferably 1 Pa-s or more; more preferably 2 Pa-s or more. Preferably, (HP)methylcellulose polymer backbone polymers have 2% solution viscosity of 30 Pa-s or less; more preferably 20 Pa-s or less; more preferably 10 Pa-s or less; more preferably 6 Pa-s or less.

In some embodiments, hydroxypropyl methylcellulose polymers are used. Among hydroxypropyl methylcellulose polymers, preferably the weight percent of hydroxypropoxyl groups is 2% or more; more preferably 4% or more; more preferably 6% or more. Preferably, the weight percent of hydroxypropoxyl groups is 20% or less; more preferably 17% or less; more preferably 14% or less. Among hydroxypropyl methylcellulose polymers, preferably the number x in structure II is 1,000 or less; more preferably 100 or less.

The cationic functional group has a positive charge under the conditions in which the method of the present invention is performed. Under different conditions, the cationic functional group may or may not be cationic. For example, the cationic functional group may contain an amine group that bears a positive charge at relatively low pH but is does not bear a positive charge at relatively high pH; in such an example, it is contemplated that the method of the present invention would be performed at sufficiently low pH that the cationic functional group bears a positive charge.

Preferred cationic functional groups contain one or more amine groups; more preferred cationic functional groups contain one or more quaternary amine groups.

Preferably, the cationic polymer has a structure that would result if one or more hydrogen atoms from one or more hydroxyl groups located on a polysaccharide polymer were replaced by a cationic functional group.

A preferred method of making the cationic polymer is to react a (HP)methylcellulose polymer with a compound of either structure III or structure IV:

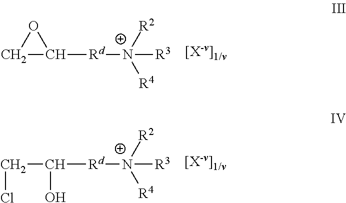

—R^{d}— is a bivalent organic group. Preferably, —R^{d}— is a hydrocarbon group with 1 to 8 carbon atoms; more preferably with 1 to 2 carbon atoms; more preferably with 1 carbon atom. —R², —R³, and —R⁴ is each independently a substituted or unsubstituted hydrocarbon group. Preferably —R², —R³, and —R⁴ are all unsubstituted hydrocarbon groups; more preferably R², R³, and R⁴ are all alkyl groups. Preferably two of R², R³, and R⁴ are methyl groups; more preferably, two of R², R³, and R⁴ are methyl groups and the third is either a methyl group or an alkyl group having 12 or more carbon atoms; more preferably, two of R², R³, and R⁴ are methyl groups and the third is an alkyl group having 12 or more carbon atoms. X^{-v} is an anion of valence v. Preferred anions are halide ions; more preferred is chloride ion.

Preferably, the cationic polymer has a structure of a polysaccharide polymer in which one or more hydroxyl hydrogen has been replaced by a cationic functional group. Preferably, the cationic polymer has Structure I, where one or more hydroxyl hydrogen has been replaced by a cationic functional group. Hydroxyl hydrogens are identified in Structure I as follows. If any of R^{a}, R^{b}, or R^{c} is a hydrogen, then that hydrogen is a hydroxyl hydrogen. If any one of R^{a}, R^{b}, or R^{c} is structure II, then the terminal hydrogen on the far right end of structure II is a hydroxyl hydrogen.

Preferably, the cationic functional group has the structure V

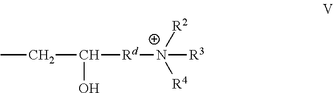

The definitions and preferences of R^{d}, R², R³, and R⁴ are discussed above.

The amount of cationic functional groups attached to the polysaccharide polymer backbone is usefully characterized by the milligrams of cationic functional group per gram of the polysaccharide polymer backbone. The weight of the cationic functional group is taken to be the total weight of all the atoms in the moiety that is bound to the polysaccharide polymer backbone and that contains the cation, excluding atoms that are present in the polysaccharide polymer backbone in the absence of the cationic functional group. Preferably the amount of cationic functional groups, in milligrams of cationic functional group per gram of polysaccharide polymer backbone, is 30 or greater; more preferably 65 or greater; more preferably 100 or greater. Preferably the amount of cationic functional groups, in milligrams of cationic functional group per gram of polysaccharide polymer backbone, is 500 or less.

The method of the present invention involves the use of a solution that contains a drug and that contains cationic polymer dissolved in water. Preferred drugs are soluble or dispersible in water at 15° C. to 40° C., in concentrations that are therapeutically useful. Preferred drugs are capable of absorption into the body through a mucosal surface; more preferably through the nasal mucosal surface.

Preferably, the volatile components present in the solution contain water. Preferably, the amount of water in the solution, by weight based on the total weight of the volatile components in the solution, is 50% or more; more preferably 75% or more; more preferably 90% or more.

The amount of polymer in the solution is preferably, by weight based on the weight of the solution, 0.01% or more; more preferably 0.1% or more. The amount of polymer in the solution is preferably, by weight based on the weight of the solution, 10% or less; 5% or less; more preferably 3% or less.

The solution optionally contains additional ingredients such as, for example, surfactants, thickeners, pH adjusters, preservatives, and mixtures thereof.

The solution may be a liquid, a gel, a lotion, a cream, or another form. Preferred is a liquid. Preferably the viscosity of the solution, as measured by steady shear viscometry using cone and plate at 10 sec⁻¹ at 25° C., is 1,000 mPa-s or less; more preferably 300 mPa-s or less; more preferably 100 mPa-s or less; more preferably 30 mPa-s or less; more preferably 10 mPa-s or less.

Preferred mucosal surface is the mucosal surface of the nasal cavity.

The following are examples of the present invention.

The materials used were as follows:
Q1=40% by weight solution in water of QUAB™ 151 epoxide: 2,3-epoxypropyltrimethylammonium chloride, from Quab Chemicals.
Q2=40% by weight solution in water of QUAB™ 360 chemical: 3-chloro-2-hydroxypropyl-cocoalkyl-dimethylammonium chloride, from Quab chemicals.
IPA=2-propanol
AD=amylodextrin: soluble starch, polymer with long chain branching, degree of polymerization approximately 100,000.
B1=50 weight % solution in water of NaOH
DIW=deionized water
HPMC1=METHOCEL™ E6, hydroxypropyl methylcellulose, from Dow Chemical Co., having methoxyl substitution of 28 to 30 weight %; hydroxypropoxyl substitution of 7 to 12 weight %, and 2% solution viscosity of 4.8 to 7.2 mPa-s at 20° C.
HPMC2=METHOCEL™ E4M, hydroxypropyl methylcellulose, from Dow Chemical Co., having methoxyl substitution of 28 to 30 weight %; hydroxypropoxyl substitution of 7 to 12 weight %, and 2% solution viscosity of 2,663 to 4,970 mPa-s at 20° C.
MC1=METHOCEL™ A15LV, methylcellulose, from Dow Chemical Co., having methoxyl substitution of 27.5 to 31.5 weight %; and 2% solution viscosity of 12-18 mPa-s at 20° C.
MC2=METHOCEL™ A4M, methylcellulose, from Dow Chemical Co., having methoxyl substitution of 27.5 to 31.5% weight %; and 2% solution viscosity of 2663 to 4970 mPa-s at 20° C.
HPMC3=METHOCEL™ F4M, hydroxypropyl methylcellulose, from Dow Chemical Co., having 2% solution viscosity of 4,000 mPa-s at 20° C.
Mucin=Mucin protein

### EXAMPLE 1: QUATERNIZATION OF BACKBONE POLYMERS WITH QUAB™ 151 EPOXIDE

Polymer backbones were dispensed into vials (1 g each). The block with vials was taken into the nitrogen-purged glove box, and 1N NaOH and DI water were added to the vials in amounts according to the table below.

Amounts in Milligrams

| Example | B1 | Q1 | IPA | DIW | Polymer (1 g each) |
|---|---|---|---|---|---|
| 1A1 | 882 | 113 | 1572 | 821 | AD |
| 1A2 | 622 | 113 | 1572 | 1071 | HPMC1 |
| 1A3 | 550 | 113 | 1572 | 1140 | HPMC2 |
| 1B1 | 882 | 452 | 1572 | 726 | AD |
| 1B2 | 622 | 452 | 1572 | 976 | HPMC1 |
| 1B3 | 550 | 452 | 1572 | 1045 | HPMC2 |
| 1C1 | 882 | 1130 | 1572 | 536 | AD |
| 1C2 | 622 | 1130 | 1572 | 786 | HPMC1 |
| 1C3 | 550 | 1130 | 1572 | 855 | HPMC2 |
| 1D1 | 622 | 113 | 1572 | 1071 | AD |
| 1D2 | 622 | 113 | 1572 | 1071 | HPMC1 |
| 1D3 | 883 | 113 | 1572 | 820 | HPMC2 |
| 1E1 | 622 | 452 | 1572 | 976 | AD |
| 1E2 | 622 | 452 | 1572 | 976 | HPMC1 |
| 1E3 | 883 | 452 | 1572 | 725 | HPMC2 |
| 1F1 | 622 | 1130 | 1572 | 786 | AD |
| 1F2 | 622 | 1130 | 1572 | 786 | HPMC1 |
| 1F3 | 883 | 1130 | 1572 | 536 | HPMC2 |

The vials were covered with a rubber mat and let sit overnight. The next day, the contents of the vials were homogenized with a spatula, forming whitish to yellowish pastes or wet powders. The block with the vials was put into a sealed, stainless steel box, and transferred to a glove box. The vials were taken from the stainless box, and charged with the amount of Q1 indicated above. Because the material in the vials was hard to agitate, 2 mL of isopropanol were added to each vial by pipette. The vials were mechanically stirred for 20 hrs at 30° C. The reactor was heated to 60° C. and stirred for another hour to react off unreacted epoxide. The reactor was cooled, the vials extracted and sealed in a steel box. In a different nitrogen-purged glove box, each vial received 2 mL of 1N NH₄Cl solution to destroy excess base.

The contents of each vial was washed into a separate dialysis bag (3,500 MWCO, SpectraPor RC), and dialyzed against DI water for approximately one week at approximately 25° C.

Dialysis bags, whose contents were low-viscosity liquids, were emptied into glass jars and devolatilized on a hot water bath, then stored on the benchtop until they were frozen in dry ice and inserted into the freeze dryer. The samples were removed from the freeze dryer and weighed. Net weights of product after freeze drying were as follows (“Ex” means Example number):

Weight of Dried Product (Grams):

| Ex. | Wt |
|---|---|
| 1A1 | 0.8375 |
| 1A2 | 0.7765 |
| 1A3 | 0.7931 |
| 1B1 | ~0.77 |
| 1B2 | 0.8314 |
| 1B3 | 0.7214 |
| 1C1 | 1.2814 |
| 1C2 | 0.8511 |
| 1C3 | 0.7599 |
| 1D1 | 0.7885 |
| 1D2 | 0.7255 |
| 1D3 | 0.8004 |
| 1E1 | 0.8790 |
| 1E2 | 0.7489 |
| 1E3 | 0.4072 |
| 1F1 | 0.8850 |
| 1F2 | 0.672 |
| 1F3 | 0/8440 |

### EXAMPLE 2: QUATERNIZATION OF BACKBONE POLYMERS WITH QUAB™ 360 EPOXIDE

Polymer backbones were dispensed into vials (1 g each). The block with vials was taken into a nitrogen-purged glove box, and a 50 weight % solution of NaOH in water (solution B1) and deionized (DI) water were added to the vials in amounts according to the table below.

The vials were let sit overnight in the glove box at approximately 25° C. The next day, the appropriate amount of QUAB™ 360 was added by Eppendorf. Extra NaOH was added by pipette in the form of a 50 weight % solution in water. The contents of the vials were homogenized with a spatula, forming whitish to yellowish pastes or wet powders. The vials were loaded into a nitrogen-purged glove box and stirred for 5 hrs at 60° C. The reactor was cooled, and the vials extracted. In the glove box, each vial received at least 2 mL of 1N NH₄Cl solution. Some vials received a minor amount of 2N HCl to reduce the pH. The vials were taken out of the box, washed or dropped into dialysis bags (3,500 MWCO, SpectraPor RC), and dialysed against DI water for approximately one week at approximately 25° C.

The contents of the dialysis bags were decanted into 100 mL vials, and put into the hot air oven (approximately 80° C.) for devolatilization. One dialysis bag with extremely viscous material was devolatilized completely on a hot water bath. The materials were frozen over dry ice and put into the freeze drier.

Amounts in Microliters

| Example | Q2 | B1 | DIW | B1⁽¹⁾ | Polymer (1 g each) |
|---|---|---|---|---|---|
| 2A1 | 572 | 37 | 3514 | 20 | AD |
| 2A2 | 403 | 26 | 3628 | 20 | HPMC1 |
| 2A3 | 357 | 23 | 3659 | 20 | HPMC2 |
| 2B1 | 2859 | 187 | 1970 | 60 | AD |
| 2B2 | 2015 | 132 | 2539 | 60 | HPMC1 |
| 2B3 | 1783 | 116 | 2696 | 60 | HPMC2 |
| 2C1 | 5717 | 373 | 39 | 100 | AD |
| 2C2 | 4030 | 263 | 1178 | 100 | HPMC1 |
| 2C3 | 3565 | 263 | 1493 | 100 | HPMC2 |
| 2D1 | 403 | 26 | 3628 | 20 | AD |
| 2D2 | 403 | 26 | 3628 | 20 | HPMC1 |
| 2D3 | 572 | 37 | 3514 | 20 | HPMC2 |
| 2E1 | 2015 | 132 | 2539 | 60 | AD |
| 2E2 | 2015 | 132 | 2539 | 60 | HPMC1 |
| 2E3 | 2861 | 187 | 1968 | 60 | HPMC2 |
| 2F1 | 4030 | 263 | 1178 | 100 | AD |
| 2F2 | 4030 | 263 | 1178 | 100 | HPMC1 |
| 2F3 | 5722 | 374 | 36 | 100 | HPMC2 |

Note
⁽¹⁾added after overnight storage

Yields are shown below.

Weight of Dried Product (Grams):

| Ex. | Wt |
|---|---|
| 2A1 | 0.8607 |
| 2A2 | 0.8004 |
| 2A3 | 0.9685 |
| 2B1 | 1.1534 |
| 2B2 | 0.8748 |
| 2B3 | 1.2073 |
| 2C1 | 1.6770 |
| 2C2 | 1.0167 |
| 2C3 | 1.7300 |
| 2D1 | 0.9156 |
| 2D2 | 0.9591 |
| 2D3 | 0.7623 |
| 2E1 | 0.7832 |
| 2E2 | 1.0581 |
| 2E3 | 1.3082 |
| 2F1 | 1.1006 |
| 2F2 | 1.2485 |
| 2F3 | 1.2947 |

### EXAMPLE 3: TESTING OF POLYMER/PROTEIN INTERACTION

A solution containing only mucin was slightly cloudy. Also, a solution was prepared of each polymer alone, and each such solution was clear.

Interaction between a polymer and the mucin protein is an indication of mucoadhesion. The interaction was visually tracked by the precipitation and settling of the polymer/protein aggregate. Test mixture solutions were prepared by mixing 500 μl of polymer solution with 500 μl of mucin solution (concentration was approximately 1% by weight) and inverting several times to mix. Images were collected after allowing solutions to settle for 1 hour at room temperature.

The test mixture solutions were evaluated for turbidity, precipitation, and phase separation of polymer that remained suspended in solution. Observation of one or more of these phenomena was considered evidence of interaction between the polymer and the mucin. The extent of interaction is reported below as an Interaction Rank, which is a comparative ranking among the samples of the extent of interaction. Test mixture solutions that had the same appearance as the mucin solution were given Interaction Rank of zero. Samples with more interaction (turbidity, precipitation, suspended phase separation, or a combination thereof) received higher Interaction Rank numbers. Samples that appeared to have the same extent of interaction as each other received the same Interaction Rank number. Result of “nt” means “not tested.”

For each combination of polymer and quaternary ammonium reactant, there were three levels of quaternary ammonium reactant, reported below as “Low,” “Med,” and “High. Example number is reported under “Ex.”

### Interaction Rank:

| | | |
|---|---|---|
| Low Q1 | Med Q1 | High Q1 |

| Polymer | Ex. | rank | Ex | Rank | Ex. | rank |
|---|---|---|---|---|---|---|
| AD | 1A1 | 3 | 1B1 | 5 | 1C1 | 5 |
| HPMC1 | 1A2 | 0 | 1B2 | 0 | 1C2 | 2 |
| HPMC2 | 1A3 | 2 | 1B3 | 3 | 1C3 | 4 |
| MC1 | 1D2 | 0 | 1E1 | nt | 1F1 | 1 |
| MC2 | 1D2 | 2 | 1E2 | 3 | 1F2 | 4 |
| HPMC3 | 1D3 | 0 | 1E3 | nt | F3 | 1 |

| | | |
|---|---|---|
| Low Q2 | Med Q2 | High Q2 |

| Polymer | Ex. | rank | Ex. | rank | Ex. | rank |
|---|---|---|---|---|---|---|
| AD | 2A1 | nt | 2B1 | nt | 2C1 | 6 |
| HPMC1 | 2A2 | 1 | 2B2 | 4 | 2C2 | 5 |
| HPMC2 | 2A3 | nt | 2B3 | nt | 2C3 | 6 |
| MC1 | 2D2 | 1 | 2E1 | 4 | 2F1 | nt |
| MC2 | 2D2 | 2 | 2E2 | 4 | 2F2 | 6 |
| HPMC3 | 2D3 | 1 | 2E3 | 3 | 2F3 | 4 |

For both the HPMC2 and MC2 polymers, the neutral polymer does exhibit a change when mixed with the mucin, but the result is much more pronounced with the cationic polymers. The HPMC2 polymer appears to be more effected by the level of substitution than MC2.

### EXAMPLE 4: EXTENT OF ATTACHMENT OF QUATERNARY AMMONIUM GROUPS

To determine the extent to which the quaternary groups attached to the polymer backbone, samples were ball milled and then subjected to combustion and elemental analysis to determine the weight fraction of nitrogen in the sample. From this, the extent of attachment of quaternary ammonium groups is assessed and reported as milligrams of structure VI per gram of backbone polymer:

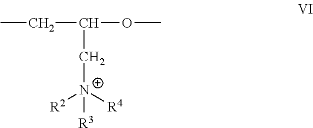

where —R², —R³, and —R⁴ are determined by the structures of Q1 and Q2.

### Extent of Attachment of Quaternary Ammonium Groups: Mgram of Structure VI Per Gram of Polymer Backbone:

| | Low Q1 | | Med Q1 | | High Q1 | |
|---|---|---|---|---|---|---|
| Polymer | Ex. | extent | Ex | extent | Ex. | extent |
| AD | 1A1 | 5.8 | 1B1 | 69.6 | 1C1 | 277.7 |
| HPMC1 | 1A2 | 3.5 | 1B2 | 25.1 | 1C2 | 81.7 |
| HPMC2 | 1A3 | 2.1 | 1B3 | 18.1 | 1C3 | 77.3 |
| MC1 | 1D1 | 2.5 | 1E1 | 23.9 | 1F1 | 92.4 |
| MC2 | 1D2 | 2.6 | 1E2 | 28.6 | 1F2 | 86.7 |
| HPMC3 | 1D3 | 2.8 | 1E3 | 28.4 | F3 | 110.6 |
| | Low Q2 | | Med Q2 | | High Q2 | |
| Polymer | Ex. | extent | Ex. | extent | Ex. | extent |
| AD | 2A1 | 5.9 | 2B1 | 69.3 | 2C1 | 341.6 |
| HPMC1 | 2A2 | 1.9 | 2B2 | 40.7 | 2C2 | 187.4 |
| HPMC2 | 2A3 | 1.7 | 2B3 | 42.3 | 2C3 | 222.2 |
| MC1 | 2D1 | 2.0 | 2E1 | 36.7 | 2F1 | 173.1 |
| MC2 | 2D2 | 49.1 | 2E2 | 39.7 | 2F2 | 210.3 |
| HPMC3 | 2D3 | 3.9 | 2E3 | 105.2 | 2F3 | 344.8 |

1. A method of delivering a drug to a mucosal surface in a living body, said method comprising applying a solution to said mucosal surface, wherein said solution comprises a cationic polymer dissolved in water, wherein said cationic polymer comprises a cationic functional group covalently attached to a polysaccharide polymer backbone selected from the group consisting of amylodextrin polymers, methylcellulose polymers, and hydroxypropyl methylcellulose polymers.

2. The method of claim 1, wherein said mucosal surface is in a nasal cavity.

3. The method of claim 1, wherein said cationic functional group comprises a quaternary ammonium cation.

4. The method of claim 1, wherein said polysaccharide polymer backbone is a hydroxypropyl methylcellulose polymer backbone.

5. The method of claim 4, wherein said hydroxypropyl methylcellulose polymer backbone has a weight percent of methoxyl groups of 15% to 40%, based on the weight of said hydroxypropyl methylcellulose polymer backbone.

6. The method of claim 4, wherein said hydroxypropyl methylcellulose polymer backbone has a weight percent of hydroxypropoxyl groups of 2% to 36%, based on the weight of said hydroxypropyl methylcellulose polymer backbone.
